(19) United States
(12) Patent Application Publication
(10) Pub. No.: US 2024/0207163 A1
(43) Pub. Date: Jun. 27, 2024

(54) RETINOL DERIVATIVE COMPOSITION AND USE THEREOF

(71) Applicants: Inertia Shanghai Biotechnology Co., Ltd., Shanghai (CN); DermaHealth Shanghai Biotechnology Co., Ltd., Shanghai (CN)

(72) Inventors: Rui YE, Shanghai (CN); Fan HU, Shanghai (CN); Xiaoyun HU, Shanghai (CN); Minhua HONG, Shanghai (CN); Yicheng XIE, Shanghai (CN)

(21) Appl. No.: 18/587,982

(22) Filed: Feb. 27, 2024

Related U.S. Application Data
(63) Continuation of application No. PCT/CN2022/115307, filed on Aug. 26, 2022.

(30) Foreign Application Priority Data
Aug. 27, 2021 (CN) 202110996638.5

(51) Int. Cl. A61K 8/67; A61Q 19/08

(52) U.S. Cl. CPC A61K 8/671; A61Q 19/08

(57) ABSTRACT

The present disclosure relates to the technical field of cosmetics and discloses a retinol derivative composition and use thereof. The present disclosure provides the retinol derivative composition, including hydroxypinacolone retinoate and retinyl propionate, wherein the ratio of the hydroxypinacolone retinoate to the retinyl propionate in parts by mass is (5-40):9. The present disclosure utilizes the synergistic effect of the hydroxypinacolone retinoate and the retinyl propionate, can extremely significantly increase the expressions of important series of proteins related to aging, and has no cytotoxicity. Therefore, when the retinol derivative composition provided by the present disclosure is used in cosmetics and/or skin care products, the composition can reach a significant anti-aging effect with a low application amount and less skin irritation.

### CROSS REFERENCE TO RELATED APPLICATIONS

The present disclosure is a continuation of PCT Patent Application No. PCT/CN2022/115307, filed Aug. 26, 2022, which claims priority to Chinese Patent Application No. 202110996638.5 filed on Aug. 27, 2021, each of which is incorporated by reference herein in its entirety.

### TECHNICAL FIELD

The present disclosure relates to the technical field of cosmetics, particularly relates to a retinol derivative composition and use thereof.

### BACKGROUND

From the age of 20-25, the skin gradually enters a natural aging state. The aging speed shows a certain difference due to different genetic genes and living habits. Early aging is not obvious shown at this time, but has a clue at the age of about 30. In addition to the multiple stresses and environmental pollution from modern society, the primary signs of the early aging are: appearance of fine lines, superficial wrinkles, and dry lines, obvious skin glycosylation, yellowing and roughness of skin, pigmentation, and dullness; reduction of the moisture content of the stratum corneum, dry skin or large pores with oil bleeding; and reduction of skin defense ability and blood circulation ability, and sensitive skin. The phenomena of the early aging are caused by various factors. With the increase of age, the subcutaneous fat layer becomes thin, the bone retracts, but the area of the skin does not change. The production rate of collagen and elastic fibers acting as a “scaffold” for the skin slows down, and therefore, the submuscular layer is hollow. The decomposition of glycosaminoglycan (i.e., sodium hyaluronate) also reduces the water retention capacity of the skin, the moisture content of the stratum corneum is reduced, the epidermis is rough and shrinks together, and obvious grooves, namely fine lines and dry lines, show. With the increase of the age, skin metabolism is too slow, and redundant sugar is dissociated in blood so as to be subjected to saccharification reaction with a protein (such as collagen) to generate advanced glycation end products (AGEs), such that the collagen loses the original elasticity, the skin turns yellow, looses, is dark, loses luster, and the wrinkle production is aggravated. In addition, excessive ultraviolet irradiation is also an important factor for skin aging: ultraviolet rays not only induce metalloprotease which accelerates the rupture of elastic fibers and collagen fibers, but also stimulate melanocytes which secrete melanin, leading to skin pigmentation, aging and roughness. Therefore, the early aging is manifested by deterioration of the whole skin, is not completely the same as the phenomena such as the increase of wrinkles and skin atrophy caused by aging, and needs to be relieved from various aspects of skin care.

Retinol (A alcohol) is the most classical anti-aging active matter, and one of the most effective recognized anti-aging substances. Its anti-aging mechanisms are: on one hand, the synthesis of collagen is increased, and on the other hand, the synthesis of dermal elastin and fibers is induced, such that fine lines and wrinkles are effectively reduced, and the firmness and elasticity of the skin are improved. However, retinol is not stable enough and is highly irritating. The high concentration of the retinol is very irritating to the skin, but low dosage of the retinol cannot reach an expected anti-aging effect, thereby limiting the wide-range application of A alcohol products. Many companies have desired to solve the above problem by searching for a retinoid substance by modifying a side chain of the retinol. However, the anti-aging effect of the retinol is often also affected by the side chain modification. For example, the common retinyl palmitate has mild irritation, but the conversion efficiency of the retinyl palmitate in vivo into the A acid which can finally start the gene expression process and play an anti-aging role is very low. The actual anti-aging effect is very weak unless a large amount of the retinyl palmitate is added. However, the addition of a large amount of the retinyl palmitate also means enhancement of the overall irritation of the cosmetics. Therefore, higher irritation of the retinol cosmetics on the market at present is difficult to avoid, no matter the pure retinol component is used or the composition of the retinol and other components is used to obtain a better anti-aging effect of the retinol.

### SUMMARY

The present disclosure aims to provide a retinol derivative composition with a strong synergistic effect. The composition can realize an extremely significant synergistic anti-aging effect with lower application amount and less skin irritation.

In order to solve the technical problems, a first aspect of the present disclosure provides the retinol derivative composition, including hydroxypinacolone retinoate and retinyl propionate, where the ratio of the hydroxypinacolone retinoate to the retinyl propionate in parts by mass is (5-40):9.

Preferably, in the retinol derivative composition provided by the present disclosure, the ratio of the hydroxypinacolone retinoate to the retinyl propionate in parts by mass is (5-20):9.

Preferably, in the retinol derivative composition provided by the present disclosure, the ratio of the hydroxypinacolone retinoate to the retinyl propionate in parts by mass is (5-10):9.

Preferably, in the retinol derivative composition provided by the present disclosure, the ratio of the hydroxypinacolone retinoate to the retinyl propionate in parts by mass is 5:9.

A second aspect of the present disclosure provides use of the retinol derivative composition according to the first aspect of the present disclosure in cosmetics and/or skin care products.

In the use provided by the present disclosure, the cosmetics and/or the skin care products are applied to the external surface of the skin.

Preferably, in the use provided by the present disclosure, the cosmetics and/or the skin care products are selected from one or more of an eye cream, a face cream, an essence, a lotion, and a facial mask essence.

Preferably, in the use provided by the present disclosure, the retinol derivative composition is used as an additive to be dispersed in a matrix raw material, and the matrix raw material is selected from one or more of an oil raw material, a powder raw material, and a colloid raw material.

Preferably, in the use provided by the present disclosure, the cosmetics and/or the skin care products further include one or more of a surfactant, an essence, a spice, a pigment, a humectant, a preservative, an antioxidant, an ultraviolet absorber, a chelating agent, an astringent, a penetration enhancer, and a nutritional additive.

Compared with the existing art, the present disclosure uses a combination of the hydroxypinacolone retinoate (HPR) and the retinyl propionate at the ratio of (5-40):9 in parts by mass, and realizes a synergistic effect. The synergistic effect is particularly remarkable when the combination of the hydroxypinacolone retinoate and the retinyl propionate at the ratio of 5:9, and the composition can extremely significantly increase the expressions of important series of proteins Col-IV, CRBPI, RARB, and Elastin related to aging, and has no cytotoxicity. Therefore, when the retinol derivative composition provided by the present disclosure is used in the cosmetics and/or the skin care products, the composition can realize a significant anti-aging effect, particularly an anti-early aging effect, with a low application amount and less skin irritation.

### BRIEF DESCRIPTION OF THE DRAWINGS

FIG. 1 shows a result of an effect on an expression of Elastin mRNA after HFF-1 cells are incubated with 40 μg/mL of HPR and 9 μg/mL of retinyl propionate alone or in combination for 24 h in an Elastin expression comparison experiment;

FIG. 2 shows a result of an effect on an expression of Elastin mRNA after HFF-1 cells are incubated with 20 μg/mL of HPR and 9 μg/mL of retinyl propionate alone or in combination for 24 h in an Elastin expression comparison experiment;

FIG. 3 shows a result of an effect on an expression of Elastin mRNA after HFF-1 cells are incubated with 10 μg/mL of HPR and 9 μg/mL of retinyl propionate alone or in combination for 24 h in an Elastin expression comparison experiment;

FIG. 4 shows a result of an effect on the expression of the Elastin mRNA after the HFF-1 cells are incubated with 5 μg/mL of the HPR and 9 μg/mL of the retinyl propionate alone or in combination for 24 h in the Elastin expression comparison experiment;

FIG. 5 shows a result of an effect on an expression of Elastin mRNA after HFF-1 cells are incubated with 2.5 μg/mL of HPR and 9 μg/mL of retinyl propionate alone or in combination for 24 h in an Elastin expression comparison experiment;

FIG. 6 shows a result of an effect on an expression of Elastin mRNA after HFF-1 cells are incubated with 1.25 μg/mL of HPR and 9 μg/mL of retinyl propionate alone or in combination for 24 h in an Elastin expression comparison experiment;

FIG. 7 shows a result of an effect on an expression of Elastin mRNA after HFF-1 cells are incubated with 0.625 μg/mL of HPR and 9 μg/mL of retinyl propionate alone or in combination for 24 h in an Elastin expression comparison experiment;

FIG. 8 shows a comparison of a multiplier of the expression of the Elastin mRNA after the HFF-1 cells are incubated with the different concentrations of the HPR and 9 μg/mL of the retinyl propionate alone or in combination for 24 h in the Elastin expression comparison experiment;

FIG. 9 shows a result of an effect on the viability of the HFF-1 cells after the HFF-1 cells are incubated with 5 μg/mL of the HPR and 9 μg/mL of the retinyl propionate alone or in combination for 24 h in a cytotoxicity experiment;

FIG. 10 shows a result of an effect on an expression of Col-IV mRNA after the HFF-1 cells are incubated with 5 μg/mL of the HPR and 9 μg/mL of the retinyl propionate alone or in combination for 24 h in a various anti-aging related mRNA expression experiment;

FIG. 11 shows a result of an effect on an expression of CRBPI mRNA after the HFF-1 cells are incubated with 5 μg/mL of the HPR and 9 μg/mL of the retinyl propionate alone or in combination for 24 h in the various anti-aging related mRNA expression experiment;

FIG. 12 shows a result of an effect on an expression of RARB mRNA after the HFF-1 cells are incubated with 5 μg/mL of the HPR and 9 μg/mL of the retinyl propionate alone or in combination for 24 h in the various anti-aging related mRNA expression experiment;

FIG. 13 shows a result of an effect on the expression of the Elastin mRNA after the HFF-1 cells are incubated with 5 μg/mL of the HPR and 9 μg/mL of the retinyl propionate alone or in combination for 24 h in the various anti-aging related mRNA expression experiment;

FIG. 14 shows a result of an effect on an expression of Col-IV mRNA after the HFF-1 cells are incubated with 5 μg/mL of the HPR and 9 μg/mL of the retinyl propionate alone or in combination for 48 h in a various anti-aging related mRNA expression experiment;

FIG. 15 shows a result of an effect on an expression of CRBPI mRNA after the HFF-1 cells are incubated with 5 μg/mL of the HPR and 9 μg/mL of the retinyl propionate alone or in combination for 48 h in the various anti-aging related mRNA expression experiment;

FIG. 16 shows a result of an effect on an expression of RARB mRNA after the HFF-1 cells are incubated with 5 μg/mL of the HPR and 9 μg/mL of the retinyl propionate alone or in combination for 48 h in the various anti-aging related mRNA expression experiment;

FIG. 17 shows a result of an effect on the expression of the Elastin mRNA after the HFF-1 cells are incubated with 5 μg/mL of the HPR and 9 μg/mL of the retinyl propionate alone or in combination for 48 h in the various anti-aging related mRNA expression experiment;

FIG. 18 shows a result of an effect on an expression of Col-IV mRNA after the HFF-1 cells are incubated with 5 μg/mL of the HPR and 9 μg/mL of the retinyl propionate alone or in combination for 72 h in a various anti-aging related mRNA expression experiment;

FIG. 19 shows a result of an effect on an expression of CRBPI mRNA after the HFF-1 cells are incubated with 5 μg/mL of the HPR and 9 μg/mL of the retinyl propionate alone or in combination for 72 h in the various anti-aging related mRNA expression experiment;

FIG. 20 shows a result of an effect on an expression of RARB mRNA after the HFF-1 cells are incubated with 5 μg/mL of the HPR and 9 μg/mL of the retinyl propionate alone or in combination for 72 h in the various anti-aging related mRNA expression experiment;

FIG. 21 shows a result of an effect on the expression of the Elastin mRNA after the HFF-1 cells are incubated with 5 μg/mL of the HPR and 9 μg/mL of the retinyl propionate alone or in combination for 72 h in the various anti-aging related mRNA expression experiment.

### DETAILED DESCRIPTION OF THE EMBODIMENTS

In order to make the objectives, technical solutions and advantages of the present disclosure clearer, the various embodiments of the present disclosure are described below in detail. However, it will be appreciated by those of ordinary skill in the art that numerous technical details are set forth in order to provide a better understanding of the present disclosure in the various embodiments of the present disclosure. But the technical solutions set forth in the present disclosure can also be realized without these technical details and various changes and modifications based on the following embodiments.

Embodiments of the present disclosure provide a retinol derivative composition including hydroxypinacolone retinoate and retinyl propionate. In some embodiments of the present disclosure, the ratio of the hydroxypinacolone retinoate to the retinyl propionate in parts by mass is (5-40):9 (i.e., the ratio of the parts by mass or mass percentage of hydroxypinacolone retinoate in the retinal derivative composition to the parts by mass or mass percentage of the retinyl propionate in the retinal derivative composition is in the range of 5:9 to 40:9). In some embodiments of the present disclosure, the ratio of the hydroxypinacolone retinoate to the retinyl propionate in parts by mass is (5-20):9. In some embodiments of the present disclosure, the ratio of the hydroxypinacolone retinoate to the retinyl propionate in parts by mass is (5-10):9. In some embodiments of the present disclosure, the ratio of the hydroxypinacolone retinoate to the retinyl propionate in parts by mass is 5:9.

The retinol derivative composition provided by the embodiments of the present disclosure can be used in cosmetics and/or skin care products, and the cosmetics and/or the skin care products are applied to the external surface of the skin. In some embodiments of the present disclosure, the cosmetics and/or the skin care products using the retinol derivative composition are selected from one or more of an eye cream, a face cream, an essence, a lotion, and a facial mask essence.

In some embodiments of the present disclosure, the retinol derivative composition is used as an additive to be dispersed in a matrix raw material, and the matrix raw material is selected from one or more of an oil raw material, a powder raw material, a colloid raw material, a grease raw material, and a solvent raw material.

The oil raw material may be any one or a combination of several of the following matrix raw materials: soybean oil, olive oil, almond oil, castor oil, peanut oil, cottonseed oil, tea seed oil, jojoba oil, avocado oil, coconut oil, palm oil, cocoa butter, rice bran oil, evening primrose oil, wheat germ oil, corn germ oil, palm wax, wood wax, mink oil, turtle oil, snake oil, egg yolk oil, beef tallow, horse tallow, lard, deer fat, lecithin, lanolin, beeswax, spermaceti wax, shellac wax, paraffin, vaseline, microcrystalline wax, ozokerite, squalane, lanolin wax, lanolin alcohol, lanolin alcohol ester, acetylated lanolin, acetylated lanolin alcohol, lanoceric acid, lanoceric acid ester, polyoxyethylene lanolin ether, polyoxyethylene lanolin alcohol ether, polyoxypropylene lanolin alcohol ether, hydrogenated lanolin, alkoxylated hydrogenated lanolin, silicone oils (e.g., dimethicone, octamethyl silicone oil, methylphenyl polysiloxane), a polysiloxane-polyoxyalkyl block copolymer, methyl hydrogen silicone oil, a Cn fatty acid and an ester thereof, and a Cn fatty alcohol and an ester thereof; where the n is ≥10, preferably 10-24, more preferably 12-20, such as 14, 16, 17, and 18. The Cn fatty acid is as follows: lauric acid, myristic acid, palmitic acid, stearic acid, oleic acid, linoleic acid, erucic acid, and capric acid. The Cn fatty alcohol is as follows: lauryl alcohol, cetyl alcohol, stearyl alcohol, isostearyl alcohol, oleyl alcohol, and coconut oil alcohol. The Cn fatty acid ester and the Cn fatty alcohol ester are as follows: isopropyl myristate, myristyl myristate, tetradecyl lactate, cetyl lactate, isopropyl palmitate, isooctyl palmitate, butyl stearate, isooctyl stearate, monoglycerol stearate, polyethylene glycol stearate, decyl oleate, glycerol caprylate, glycerol caprate, and glycerol trioleate.

The powder raw material may be any one or a combination of several of the following matrix raw materials: mica, magnesium carbonate, titanium dioxide, zincite powder, calcium hydroxide, calcium carbonate, kaolin, calcium stearate, zinc stearate, magnesium stearate, talcum powder, silicon dioxide, aluminum hydroxide, calcium pyrophosphate, calcium bicarbonate, and bentonite.

The grease raw material may be any one or more of a vegetable oil raw material, an animal oil raw material, a mineral oil raw material, a synthetic oil raw material, and a semisynthetic oil raw material For example, the colloid raw material may be any one or a combination of several of the following matrix raw materials: starch, cyclodextrin, xanthan gum, carrageenan, guar gum, arabic gum, tragacanth gum, agar, shellac, sodium alginate, gelatin, methyl cellulose, ethyl cellulose, sodium carboxymethyl cellulose, hydroxyethyl cellulose, hydroxypropyl cellulose, a polycellulose ether quaternary ammonium salt, polyvinyl alcohol, polyvinylpyrrolidone, sodium polyacrylate, polyoxyethylene, a vinylpyrrolidone/vinyl acetate copolymer, carbomer resin, magnesium aluminum silicate, and C12-15 alkyl benzoate.

The solvent raw material may be any one or a combination of several of the following matrix raw materials: water, ethanol, propanol, isopropanol, isobutanol, ethylene glycol, 1,2-propylene glycol, cyclohexane, dichlorodifluoromethane, tetrafluorodichloroethane, ethylene glycol monoethyl ether, diethylene glycol monoethyl ether, acetone, methyl isobutyl ketone, ethyl acetate, butyl acetate, amyl acetate, and dibutyl phthalate.

In some embodiments of the present disclosure, the cosmetics and/or the skin care products using the retinol derivative composition further include one or more of a surfactant, an essence, a spice, a pigment, a humectant, a preservative, an antioxidant, an ultraviolet absorber, a chelating agent, an astringent, a penetration enhancer, a pH regulator, and a nutritional additive.

The surfactant may be: 1) an anionic surfactant, such as C12-14 fatty alcohol ammonium sulfate, C12-14 fatty alcohol polyoxyethylene ether carboxylate, diethanolamine lauryl sulfate, C12-14 fatty alcohol polyoxyethylene ether ammonium sulfate, triethanolamine lauryl sulfate, an alcohol ether phthalic acid monoester sodium salt, acylated peptide, a dodecanoyl monocthanolamide sulfate sodium salt, n-octyl alcohol polyoxypropylene ether succinate monoester sulfonate, cetyl alcohol succinate monoester sulfonate, a sulfo succinate monoester disodium salt, a succinate monooctadecylester disodium salt, an alcohol ether sulfo succinate monoester disodium salt, a fatty alcohol polyoxyethylene ether (sodium) sulfo succinate monoester ammonium salt, an olcamide sulfo succinate monoester disodium salt, an N-acyl glutamate potassium salt, sodium pyrrolidone carboxylate, sodium polyacrylate, fatty alcohol polyoxyethylene cther phosphate ester, fatty alcohol polyoxyethylene ether phosphate monoester and a salt thereof, a fatty alcohol polyoxyethylene ether phosphate monoester ethanolamine salt, alkylphenol polyoxyethylene ether phosphate monoester and a salt thereof, a nonylphenol ether phosphate monoester ethanolamine salt, an alkyl phosphate salt, a phthalic acid monolauryl ester salt, zinc undecylenate, an anionic amino acid surfactant, N-C12-18 acyl sodium glutamate, sodium N-lauroyl-L-alanine, monosodium N-stearoyl glutamate, monosodium N-cocoyl glutamate, monosodium N-mixed fatty acyl glutamate, and long straight-chain alkyl aryl ether sodium sulfonate TH; 2) a nonionic surfactant such as: polyoxyethylene olcyl ether (e.g. emulsifier VO series), alkylphenol polyoxyethylene ether (e.g. emulsifier OPE series), polyoxyethylene stearate, cthylene glycol monostearate, ethylene glycol distearate, polyethylene glycol monostearate, polyethylene glycol distearate, polyethylene glycol dilaurate, glyceryl distearate, glyceryl tristearate, sucrose stearate, polyoxyethylene glyceryl cther monostearate, N-lauroyl glutamate dialkanol ester, fatty alcohol benzoate, sorbitan fatty acid ester (Span, e.g. Span-20, Span 60, Span 65, Span-80, Span-83, and Span-85), polyoxyethylene sorbitan fatty acid ester (Tween, e.g. Tween-20, Tween-40, Tween-60, Tween-61, and Tween-80), diethanolamine, triethanolamine, fatty acid monocthanolamide (such as coconut oil monocthanolamide, lauric acid monocthanolamide, and palmitic acid monocthanolamide), coconut oil diethanolamide, lauric acid dicthanolamide, methyl glucose sesquistearate, polyoxyethylene methyl glucoside stearate, ethylene glycol glucoside stearate, propylene glycol glucoside stearate, C8-16 alkyl glucoside, glycerol glucoside stearate, succinate lauryl ether monoester, glyceride polyoxyethylene lauryl ether, mannitol stearate ester, polyoxyethylene mink oil, mink oil methyl ester, and mink oil isopropyl ester; 3) a cationic surfactant such as C12-18 alkyltrialkylammonium chloride (e.g. cetyldimethylammonium chloride, octadecyltrimethylammonium chloride, and cetyltrimethylammonium chloride), olcamidopropyl dihydroxypropyl dimcthylammonium chloride, dihydroxypropyl dimethyldodecylammonium chloride, a dimethyldiallylammonium chloride/acrylamide copolymer, olcamidopropyl-2,3-dihydroxypropyl dimethylammonium chloride, olcamidopropyl dimethylethylammonium ethylsulfate, di-C12-18 alkyl dimethylammonium chloride, a polyquaternium-11 conditioner, an M-505 polyquaternium-7 conditioner, mink oil amidopropyl amine-chitosan, mink oil amidopropyl amine-protein hydrolysate, cationic protein peptide, guar gum-hydroxypropyl trimethyl ammonium chloride, a polycellulose ether quaternary ammonium salt, triethanolamine monooleate, and DNP series; and 4) a zwitterionic surfactant such as C12-18 alkyl dihydroxyethyl betaine, N-C12-18 alkyl-N-(2-hydroxyethyl)-N-(2-carboxamidocthyl)ammonium acetate, C12-18 alkoxyhydroxypropyl betaine, cocamidopropylamine oxide, sodium cocoyl isethionate, octadecyldimethylamine oxide, cocamidodiethanolamine oxide, N-alkyl-β-aminopropionyldiethanolamine, lauroylpropylamine oxide, N-C12-18 acyl glutamic acid, hydroxyethyldecanoic imidazoline betaine, hydroxyethylmyristic imidazoline betaine, and hydroxyethylpalmitic imidazoline betaine.

The essence and the spice may be selected from: 1) a natural spice such as wormwood oil, eucalyptus oil, star anise oil, lime oil, a white lilac extract, clove oil, michelia alba oil, a michelia alba extract, michelia alba leaf oil, cedar oil, peppermint oil, atractylis oil, a sweet clover extract, petitgrain oil, oleum ocimi gratissimi, a jasminum grandiflorum extract, wintergreen oil, angelica oil, an angelica extract, lichenic acid, gaultheria forrestii oil, seville orange flower oil, seville orange leaf oil, a hyacinth extract, a liquidambar formosana extract, cinnamomum camphora oil, sassafras oil, spikenard oil, an osmanthus fragrans extract, tangerine oil, carrot seed oil, castoreum, ambrette seed oil, michelia champaca flower oil, palchouli oil, Artemisia oil, murraya daniculata concrete, Cassie Concrete, ginger oil, mandarin oil, peppermint oil, a chrysanthemum extract, cumin oil, ambergris, spearmint oil, civet, wintersweet oil, a convallaria keiskei extract, costus root oil, aglaia odorata oil, geranium oil, rose oil, a jasmine extract, oyster oil, a crimson glory rose extract, crimson glory rose oil, eucalyptus citriodora leaf oil, lemon oil, a qilixiang extract, phoebe oil, celery seed oil, nutmeg oil, cinnamon oil, litsea cubeba oil, mink balm tincture, a narcissus extract, pine needle oil, a tree moss extract, a jasminum grandiflorum extract, musk, muskrat balm, sweet orange oil, sandalwood oil, tuberose oil, a tuberose extract, marigold essential oil, fennel oil, cypress oil, citronella oil, geranium oil, carnation oil, perilla oil, clary sage oil, Cyperus rotundus oil, bergamot mint oil, vetiver oil, elsholtzia ciliata oil, an elsholtzia ciliata extract, rosa rubus oil, a rosa rubus extract, lavender oil, bay leaf oil, ylang oil, coriander seed oil, mentha rotundifolia oil, a labdanum extract, cinnamomum petrophilum oil, pomelo oil, iris oil, nicotiana alata oil, citron leaf oil, a gardenia flower extract, amorpha frutcosa oil, a violet leaf extract, guaiacol, 4-methyl guaiacol, 4-ethyl guaiacol, natural vanillin, and camphor; 2) a synthetic essence such as limonene, longifolene, β-caryophyllene, isolongifolene, isolongifolenone, isolongifolanone, bromostyrol, diphenylmethane, diphenyl ether, m-methyl diphenyl ether, eugenol, isocugenol, β-naphthyl methyl ether, β-naphthyl ethyl ether, p-methyl anisole, isoeugenol benzyl ether, rose oxide, 7-methoxy-3,7-dimethyloctan-2-ol, 4-methylphenyl benzyl ether, ambroxan, methyl cedryl ether, epoxycedrane, elintaal, elintaal N, 3-hexenol, decanol, lauryl alcohol, benzyl alcohol, phenethyl alcohol, p-methoxybenzyl alcohol, cinnamyl alcohol, dimethyl benzyl alcohol, geraniol, nerol, linalool, rhodinol, terpineol, citronellol, racemic menthol, menthol, borneol, cedrol, cedranone, methyl cedranone, 3,4-dioxymethylene benzyl alcohol, a-methyl-3,4-dihydromethylenebenzenepropanal, 2-tert-butyl-4-methylcyclohexanol, sclareol, santalol, 3-methyl-5-(2′,2′,3′-trimethylcyclopenten-1′-yl)pent-2-ol, benzaldehyde, acetophenone, phenylacetic acid, p-methylacetophenone benzophenone, lauraldehyde, cinnamaldehyde, cinnamic acid, α-pentyl-β-phenylacrolein, α-phenylpropionaldehyde, lilial, lyral, cyclamen aldehyde, 3,4-dioxymethylene benzaldehyde, 3,7-dimethyl-6-octenal, 3,7-dimethyl-7-hydroxyoctanal, citral, β-cyclohomocitral, Vertoliff, myrac aldehyde, 4-hydroxy-3-methoxybenzaldehyde, α-dihydrodamascone, β-damascenone, ionone, 6-methylionone, norone, carvone, piperonylacetone, raspberry ketone, 3,3-Dimethyl-1-acetylcyclohexane, cis-jasmone, heptanal glycol acetal, phenylacetaldehyde dimethyl acetal, octanal dimethyl acetal, α-amyl cinnamic aldehyde dimethyl acetal, citral dimethyl acetal, citral diethyl acetal, anisaldehyde dimethyl acetal, 2-(α-methylbenzyl)-4-methyl-1,3-dixolane, acetaldehyde benzyl cthyl acetal, [2-(1-ethoxyethoxy)ethyl]-benzene, fructone, 4-tert-butylcyclohexanone ethylene ketal, geranyl formate, citronellyl formate, aphermate, cis-3-Hexenyl Acetate, benzyl acetate, phenylethyl acetate, cinnamyl acetate, terpinyl acetate, bornyl acetate, linalyl acetate, geranyl acetate, p-tert-butylcyclohexyl acetate, o-tert-butylcyclohexyl acetate, vetiveryl acetate, succinyl acetate, rosamusk, decahydronaphthyl acetate, cedryl acetate, geranyl propionate, trichloromethylbenzyl acetate, benzyl butyrate, cis-3-hexenyl butyrate, geranyl butyrate, phenethyl 2-methylpentanoate, p-cresyl isobutyrate, methyl 2-nonenoate, ethyl benzoate, methyl benzoate, cis-3-hexenyl benzoate, benzyl benzoate, ethyl phenylacetate, p-cresyl phenylacetate, ethyl cinnamate, butyl salicylate, isobutyl salicylate, isoamyl salicylate, cis-3-hexenyl salicylate, hexyl salicylate, benzyl salicylate, phenethyl salicylate, cyclohexyl salicylate, jasmonate, jasMonyl, methyl 2-pentylcyclopentanone acetate, aurantiol, γ-nonanolactone, γ-unsecalactone, coumarin, indole, 3-methylindole, 3,7-dimethyl-2,6-octadienenitrile, p-methoxybenzonitrile, musk xylene, musk ketone, 2,6-dinitro-3-methoxyl-4-tert-butyl toluene, 1,1-dimethyl-4-acetyl-6-tert-butyl indane, hexamethyl tricyclic isochroman musk, 1,1,2,3,3,6-hexamethyl-5-acetyl indane, musk-T, musk-M, musk-L, musk-F, 11-oxahexadecanolide, and tonalid; and 3) an essence such as rose essence, jasmine essence, sandalwood essence, and brandy essence.

The pigment may be selected from one or a combination of several of the following components: zirconium dioxide, lead acetate, silver nitrate, chlorophyll, copper chlorophyll, iron ferrocyanide, guanine, fast red, food red I, vat red I, carmine, edible waxberry red, edible cherry red, food red 17, amaranth, fast green, food yellow 3, edible lemon yellow, edible Sudan yellow, edible indigo, food blue 2, fluorescent peach red, β-carotene, henna, gardenia red pigment, gardenia green pigment, gardenia blue pigment, gardenia yellow pigment, mica titania pearlescent pigment, iron oxide red, iron oxide yellow, iron oxide black, chromium oxide green, bismuth oxychloride, basic peach red, vermilion R, quinoline yellow, shikonin, lac dyer red pigment, ultramarine, acid red 87, acid green 25, acid fluorescent yellow, acid orange 7, bronze red 53, lithol rubine BK, lithol rubine 2R, and everlasting orange.

The preservative may be selected from one or a combination of several of the following components: sorbic acid and a salt thereof, propionate, paraben, benzyl alcohol, benzoic acid and a salt thereof, 5-chloro-2-methyl-4-isothiazolin-3-one, 2-methyl-4-isothiazolin-3-one, Casson, imidazolidinyl urea, dehydroacetic acid and a salt thereof, sodium sulfite, sodium bisulfite, sodium metabisulfite, 2,2-bromo-2-nitro-1,3-propanediol (bronopol), isoascorbic acid and a salt thereof, kojic acid and an ester thereof, azelaic acid, usnic acid, and resorcinol.

The antioxidant may be selected from one or a combination of several of the following components: SOD, butyl hydroxy anisole, gallate, ascorbic acid and a salt thereof, isoascorbic acid and a salt thereof, sitosterol, rutin, and tocopherol.

The humectant may be selected from one or a combination of several of the following components: 1,2-butanediol, sorbitol, xylitol, glycerol, lactate, polyethylene glycol, DL-pyrrolidone carboxylate, D-glucose, kojic acid and an ester thereof, uric acid, orotic acid, pectic acid, laminine, collagen, trehalose, and 12-cholesteryl hydroxystearate.

The ultraviolet absorber may be selected from one or a combination of several of the following components: pyridoxine hydrochloride, kojic acid and an ester thereof, rutin, barbaloin, caffeic acid, hesperetin, 2-ethylhexyl salicylate, 2-ethylhexyl p-dimethylaminobenzoate, 2-ethylhexyl p-methoxycinnamate, phenyl salicylate, Ethyl anthranilate, 2-hydroxy-4-methoxybenzophenone, 2-hydroxy-4-methoxybenzophenone-5-sulfonic acid, and 2-hydroxy-4-octyloxybenzophenone.

The chelating agent may be selected from one or a combination of several of the following components: an EDTA disodium salt, zinc dipyridyl thione, and sodium thiosulfate.

The astringent may be selected from one or a combination of several of the following components: zinc p-phenolsulfonate, aluminum hydroxychloride, aluminum sulfate, potassium aluminum sulfate, zinc sulfate, aluminum chloride, zinc chloride, and calamine.

The penetration enhancer may be selected from one or a combination of several of the following components: dimethyl sulfoxide, 1-n-dodecylazacyclohept-2-one, and 2-aminoethanol.

The pH regulator may be selected from one or a combination of several of the following components: tartaric acid, lactic acid, boric acid, and succinic acid.

The nutritional additive may be selected from one or a combination of several of the following components: chitin, chitosan, silk peptide, fibroin, lecithin, epidermal cell growth factor, trehalose, lactose, pearl powder, collagen, collagen hydrolysate, hyaluronic acid and a salt thereof, egg membrane element, phytosterol, lysozyme chloride, sodium chondroitin sulfate, royal jelly, propolis, honey, glycyrrhizic acid, glycyrrhetinic acid, γ-linolenic acid, kojic acid and an ester thereof, gibberellic acid, glycine, L-aspartic acid and a salt thereof, L-cystine and a salt thereof, DL-alanine, L-serine, DL-serine, L-methionine, DL-methionine, L-lysine hydrochloride, L-threonine, inositol, maltol, pantothenate, D-panthenol, retinol acetate, allantoin, pyridoxine hydrochloride, cortisone, nicotinamide, vitamins, estrogen, hematoxylin, uric acid, orotic acid, aloin, azelaic acid, abictic acid, usnic acid, laminine, thymosin, alizarin, limonene, baicalein, deoxycholic acid, guaiacin, hesperetin, tannic acid, selenium disulfide, theasaponin, triethyl citrate, butyl hydroxy toluene, phytic acid, and poly(dimethyl diallyl ammonium chloride).

### Experimental Example

(The instruments, kits, cells, and reagents used in the experimental example were publicly commercially available and were commonly used in the art. The operations of the instruments and the kits are performed according to the instructions.)

Elastin mRNA Expression Comparison Experiment

1.1 Experimental content: an effect of the different concentrations of HPR and 9 μg/mL of retinyl propionate alone or in combination on an expression of Elastin mRNA in HFF-1 cells.

1.2 Experimental method: the HFF-1 cells were seeded in a 96-well plate (100,000 cells/mL and 2 mL per well) and administrated in different-concentration HPR groups, 9 μg/mL retinyl propionate groups, and different-concentration HPR+9 μg/mL retinyl propionate combined groups (specific parameters of each experimental group were shown in Table 1). A blank control was set at the same time. Total RNA of the cells was extracted at 24 h and the expression of the Elastin mRNA was detected by qPCR.

TABLE 1

| | HPR | | Retinyl | |
|---|---|---|---|---|
| | group | | propionate group | Combined group |
| Elastin expression | 40 | μg/mL | 9 μg/mL | 40 μg/mL of HPR + 9 μg/mL |
| experimental group 1 | | | | of retinyl propionate |
| Elastin expression | 20 | μg/mL | 9 μg/mL | 20 μg/mL of HPR + 9 μg/mL |
| experimental group 2 | | | | of retinyl propionate |
| Elastin expression | 10 | μg/mL | 9 μg/mL | 10 μg/mL of HPR + 9 μg/mL |
| experimental group 3 | | | | of retinyl propionate |
| Elastin expression | 5 | μg/mL | 9 μg/mL | 5 μg/mL of HPR + 9 μg/mL |
| experimental group 4 | | | | of retinyl propionate |
| Elastin expression | 2.5 | μg/mL | 9 μg/mL | 2.5 μg/mL of HPR + 9 μg/mL |
| experimental group 5 | | | | of retinyl propionate |
| Elastin expression | 1.25 | μg/mL | 9 μg/mL | 1.25 μg/mL of HPR + 9 μg/mL |
| experimental group 6 | | | | of retinyl propionate |
| Elastin expression | 0.625 | μg/mL | 9 μg/mL | 0.625 μg/mL of HPR + 9 μg/mL |
| experimental group 7 | | | | of retinyl propionate |

The steps of extracting the total RNA of the cells and detecting the expression of the Elastin mRNA by qPCR were completed by a kit conventionally used by a person skilled in the art. Each operation step was performed referring to the instructions of the kit.

RNA extraction: a cell sample was taken, a supernatant was removed, 1 mL of a TRIZON reagent was added into each well of a 6-well plate, the cells were stood, cracked, and transferred to a 1.5-mL EP tube, 0.2 mL of chloroform was added, and the mixture was shaken, uniformly mixed, stood on ice, and centrifuged at 12,000 rpm/min at 4° C. for 15 min; about 400 μL of an obtained supernatant was taken, isopropanol with the same volume was added, the mixture was lightly shaken, stood on ice at 4° C., and centrifuged at 12,000 rpm/min for 10 min; an obtained supernatant was removed, 1 mL of 75% ethanol was added into an obtained precipitate, an EP tube was shaken to float the precipitate to achieve a washing effect, the obtained product was centrifuged at 7,500 rpm/min at 4° C. for 10 min; an obtained supernatant was removed, a part of ethanol was left, the product was centrifuged at 7,500 rpm/min at 4° C. for 5 min, and residual ethanol was removed from the inner wall of the tube; and an obtained supernatant was removed, the liquid was aspirated, and a certain amount of RNA-free water was added for resuspension and dissolution. 2 μL of the product was analyzed on a SpectraMax iD3 multifunctional microplate reader for detecting the RNA concentration and A260/A280 value.

cDNA synthesis: the mRNA was subjected to reverse transcription into cDNA by using a HiFiScript cDNA synthesis kit.

Fluorescence quantification: the expression of the Elastin mRNA in the cell sample was detected by mixing an UltraSYBR Mixture premixed solution and the cDNA, and loading the mixture into a machine.

1.3 Experimental results: data was input by the Excel, subjected to statistical processing by a Prism (Version 7) software package, and expressed as mean±standard error. Each data was subjected to a one-way analysis of variance and p<0.05 was considered to be statistically different.

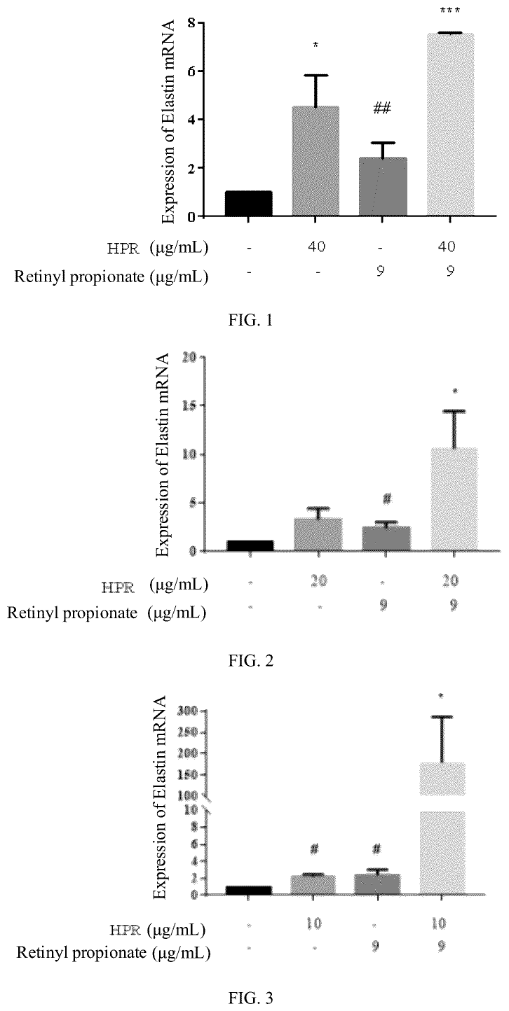

FIG. 1 showed a result of the effect on the expression of the Elastin mRNA after the HFF-1 cells were incubated with 40 μg/mL of HPR and 9 μg/mL of retinyl propionate alone or in combination for 24 h in the Elastin expression comparison experiment;

FIG. 2 showed a result of the effect on the expression of the Elastin mRNA after the HFF-1 cells were incubated with 20 μg/mL of the HPR and 9 μg/mL of the retinyl propionate alone or in combination for 24 h in the Elastin expression comparison experiment;

FIG. 3 showed a result of the effect on the expression of the Elastin mRNA after the HFF-1 cells were incubated with 10 μg/mL of the HPR and 9 μg/mL of the retinyl propionate alone or in combination for 24 h in the Elastin expression comparison experiment;

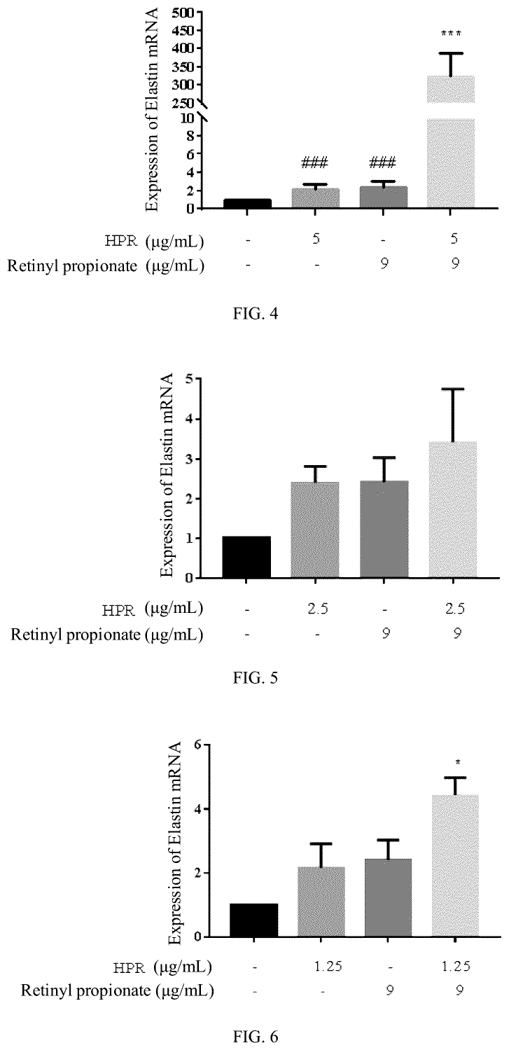

FIG. 4 showed a result of the effect on the expression of the Elastin mRNA after the HFF-1 cells were incubated with 5 μg/mL of the HPR and 9 μg/mL of the retinyl propionate alone or in combination for 24 h in the Elastin expression comparison experiment;

FIG. 5 showed a result of the effect on the expression of the Elastin mRNA after the HFF-1 cells were incubated with 2.5 μg/mL of the HPR and 9 μg/mL of the retinyl propionate alone or in combination for 24 h in the Elastin expression comparison experiment;

FIG. 6 showed a result of the effect on the expression of the Elastin mRNA after the HFF-1 cells were incubated with 1.25 μg/mL of the HPR and 9 μg/mL of the retinyl propionate alone or in combination for 24 h in the Elastin expression comparison experiment; and

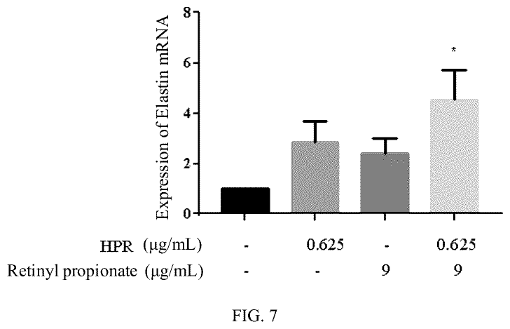

FIG. 7 showed a result of the effect on the expression of the Elastin mRNA after the HFF-1 cells were incubated with 0.625 μg/mL of the HPR and 9 μg/mL of the retinyl propionate alone or in combination for 24 h in the Elastin expression comparison experiment.

(In FIGS. 1-7: n=3, Mean±SEM. “*” indicated p<0.05 compared to the control group, “**” indicated p<0.01 compared to the control group, and “***” indicated p<0.001 compared to the control group; and “#” indicated p<0.05 compared to the HPR+retinyl propionate group, “##” indicated p<0.01 compared to the HPR+retinyl propionate group, and “###” indicated p<0.001 compared to the HPR+retinyl propionate group.)

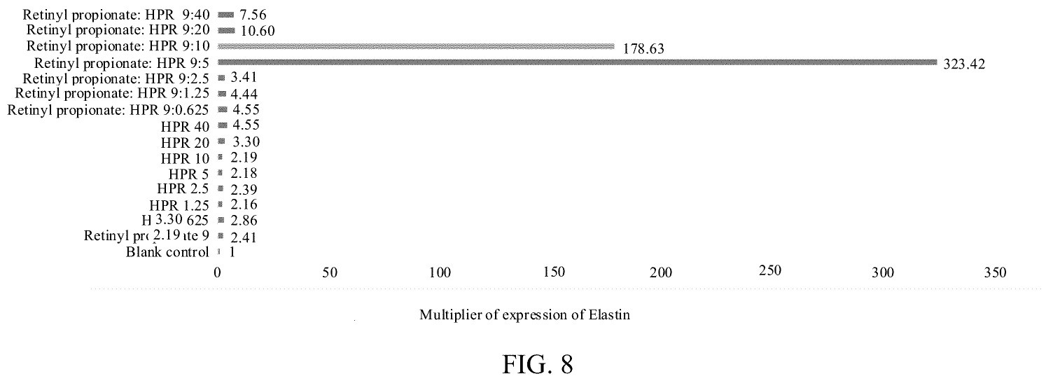

FIG. 8 showed a comparison of a multiplier of the expression of the Elastin mRNA after the HFF-1 cells were incubated with the different concentrations of the HPR and 9 μg/mL of the retinyl propionate alone or in combination for 24 h in the Elastin expression comparison experiment.

FIGS. 1-4 and 8 showed that when the cells were administrated with the hydroxypinacolone retinoate and the retinyl propionate at a ratio of 5:9 to 40:9 in parts by mass for 24 h, the combined administration can obviously promote the expression of the Elastin mRNA compared with the single administration.

FIGS. 5-7 and 8 showed when the cells were administrated with the hydroxypinacolone retinoate and the retinyl propionate at other ratios in parts by mass for 24 h, the combined administration did not obviously promote the expression of the Elastin mRNA compared with the single administration.

### Cytotoxicity Experiment

2.1 Experimental content: an effect of 5 μg/mL of HPR and 9 μg/mL of retinyl propionate alone or in combination on the viability of HFF-1 cells.

2.2 Experimental method: the HFF-1 cells were seeded in a 96-well plate (3,000 cells/mL and 100 μL per well) and administrated in a 5 μg/mL HPR group, a 9 μg/mL retinyl propionate group, and a 5 μg/mL HPR+9 μg/mL retinyl propionate group. A blank control was set at the same time. The cell viability after 24 h of the administration treatment was detected using a CCK-8 kit.

2.3 Experimental results: data was input by the Excel, subjected to statistical processing by a Prism (Version 7) software package, and expressed as meantstandard error. Each data was subjected to a one-way analysis of variance and p<0.05 was considered to be statistically different. Data of the cytotoxicity experiment was shown in Table 2.

TABLE 2

| Blank | 5 μg/mL of | 9 μg/mL of retinyl | 5 μg/mL of HPR + 9 μg/mL |
|---|---|---|---|
| control | HPR | propionate | of retinyl propionate |
| 100 | 104.914144 | 100.1752163 | 143.8121681 |
| 100 | 101.4125131 | 100.8383427 | 144.8877268 |
| 100 | 100.3369545 | 95.66272205 | 145.1545947 |

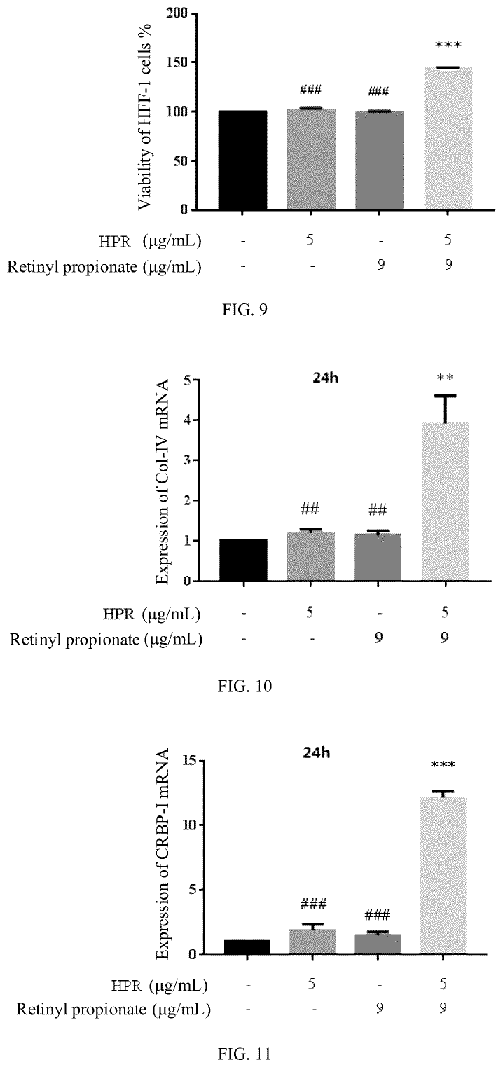

FIG. 9 showed a result of the effect on the viability of the HFF-1 cells after the HFF-1 cells were incubated with 5 μg/mL of the HPR and 9 μg/mL of the retinyl propionate alone or in combination for 24 h. (In FIG. 9: n=3, Mean±SEM. “*” indicated p<0.05 compared to the control group, “**” indicated p<0.01 compared to the control group, and “***” indicated p<0.001 compared to the control group; and “###” indicated p<0.001 compared to the HPR+retinyl propionate group.) The results showed that after the administration treatment for 24 h, 5 μg/mL of the HPR+9 μg/mL of the retinyl propionate can promote the growth of the HFF-1 cells with the effect more significant than that of the single administration (p<0.001).

Various Anti-Aging Related mRNA Expression Experiment

3.1 Experimental content: an effect of 5 μg/mL of HPR and 9 μg/mL of retinyl propionate alone or in combination on expressions of collagen-related mRNAs in HFF-1 cells.

3.2 Experimental method: the HFF-1 cells were seeded in a 96-well plate (3,000 cells/mL and 2 mL per well) and administrated in a 5 μg/mL HPR group, a 9 μg/mL retinyl propionate group, and a 5 μg/mL HPR+9 μg/mL retinyl propionate group. A blank control was set at the same time. Total RNA of the cells was extracted at 24 h, 48 h, and 72 h and the expressions of the collagen-related mRNAs were detected by qPCR. (The specific operation steps referred to section 1.2)

3.3 Experimental results: data was input by the Excel, subjected to statistical processing by a Prism (Version 7) software package, and expressed as meantstandard error. Each data was subjected to a one-way analysis of variance and p<0.05 was considered to be statistically different. The data of the expression of each anti-aging related mRNA was respectively shown in Tables 3-5:

TABLE 3
mRNA expressions after 24 h

| | Blank | 5 μg/mL of | 9 μg/mL of retinyl | 5 μg/mL of HPR + 9 μg/mL |
|---|---|---|---|---|
| | control | HPR | propionate | of retinyl propionate |
| Col IV | 1 | 1.356604327 | 1 | 4.40762046 |
| | 1 | 1.01395948 | 1.35660433 | 4.78991482 |
| | 1 | 1.222640278 | 1.07922824 | 2.5668518 |
| CRBPI | 1 | 1.596707444 | 1.04246576 | 12.5533456 |
| | 1 | 3.22276725 | 1.58008262 | 11.1579493 |
| | 1 | 1.094293701 | 1.86606598 | 12.7285837 |
| RARB | 1 | 1.079228237 | 1.30134186 | 5.0280535 |
| | 1 | 3.732131966 | 1.21419488 | 4.05583792 |
| | 1 | 5.063026376 | 2.82842712 | 4.1410597 |
| Elastin | 1 | 1.777685362 | 1.77768536 | 106.152902 |
| | 1 | 14.52030649 | 16.1112888 | 243.875327 |
| | 1 | 1.029040669 | 1.15668818 | 72.5045687 |

TABLE 4
mRNA expressions after 48 h

| | Blank | 5 μg/mL of | 9 μg/mL of retinyl | 5 μg/mL of HPR + 9 μg/mL |
|---|---|---|---|---|
| | control | HPR | propionate | of retinyl propionate |
| Col IV | 1 | 1.474269 | 1.385109 | 3.24901 |
| | 1 | 1.094294 | 1.484524 | 4.531536 |
| | 1 | 1.94504 | 1.117287 | 2.770219 |
| CRBPI | 1 | 1.580083 | 1.647182 | 32 |
| | 1 | 1.094294 | 1.274561 | 28.44297 |
| | 1 | 1.488129 | 1.647182 | 32.4467 |
| RARB | 1 | 1.658639 | 1.301342 | 4.469149 |
| | 1 | 1.189207 | 1.60214 | 5.278032 |
| | 1 | 3.605002 | 1.079228 | 5.656854 |
| Elastin | 1 | 2.928171 | 1.866066 | 903.8878682 |
| | 1 | 2.80889 | 2.770219 | 1351.176101 |
| | 1 | 3.317278 | 3.010493 | 797.8645296 |

TABLE 5
mRNA expressions after 72 h

| | Blank | 5 μg/mL of | 9 μg/mL of retinyl | 5 μg/mL of HPR + 9 μg/mL |
|---|---|---|---|---|
| | control | HPR | propionate | of retinyl propionate |
| Col IV | 1 | 1.164734 | 1.624505 | 4.40762046 |
| | 1 | 1.265055 | 1.189207 | 3.05251842 |
| | 1 | 0.939523 | 0.97942 | 3.73213197 |
| CRBPI | 1 | 3.295049 | 1.071773 | 31.3414495 |
| | 1 | 4.016949 | 3.271608 | 32 |
| | 1 | 1.79005 | 1.086735 | 39.9465776 |
| RARB | 1 | 2.394957 | 1.802501 | 9.78112222 |
| | 1 | 1.042466 | 1.164734 | 4.22807216 |
| | 1 | 4.924577653 | 1.301342 | 6.02098699 |
| Elastin | 1 | 1.962782 | 5.735821 | 1964.57292 |
| | 1 | 3.519572 | 6.727171 | 1351.1761 |
| | 1 | 4.112455 | 1.274561 | 1951.00262 |

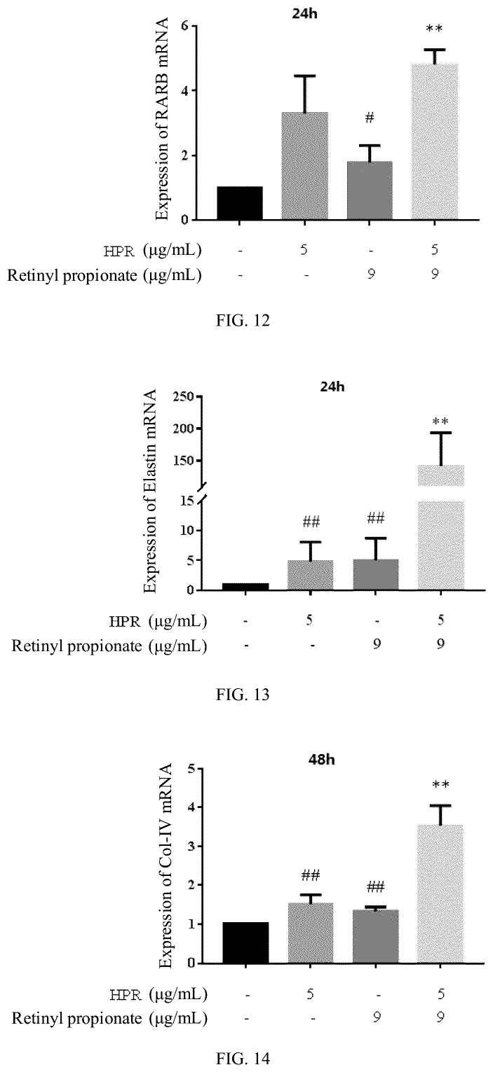

FIGS. 10-13 successively showed results of the effect on the expressions of the Col-IV mRNA, the CRBPI mRNA, the RARB mRNA, and Elastin mRNA after the HFF-1 cells were incubated with 5 μg/mL of the HPR and 9 μg/mL of the retinyl propionate alone or in combination for 24 h.

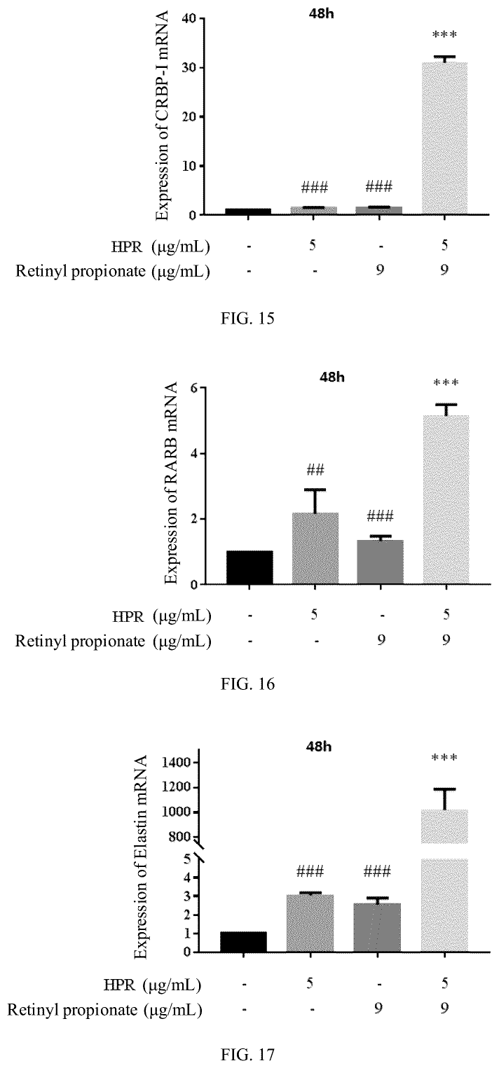

FIGS. 14-17 successively showed results of the effect on the expressions of the Col-IV mRNA, the CRBPI mRNA, the RARB mRNA, and Elastin mRNA after the HFF-1 cells were incubated with 5 μg/mL of the HPR and 9 μg/mL of the retinyl propionate alone or in combination for 48 h.

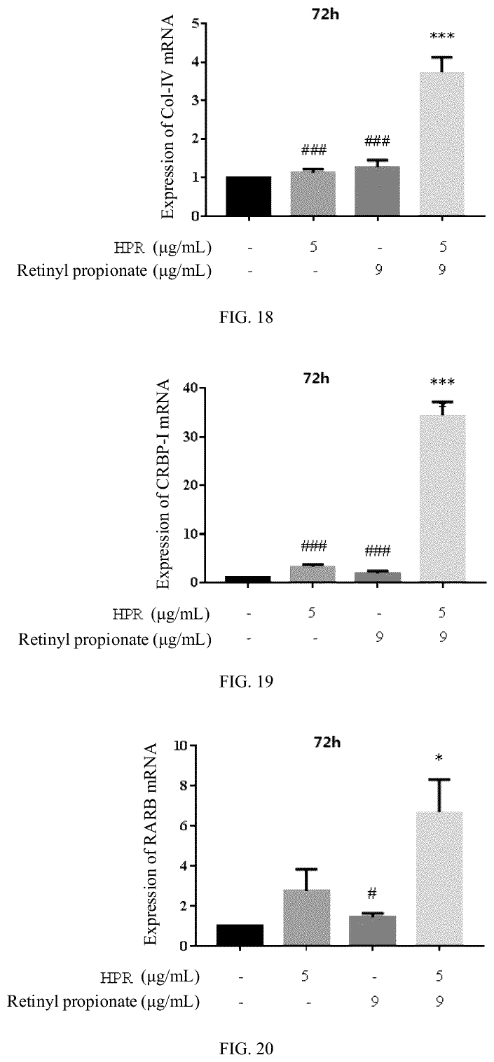

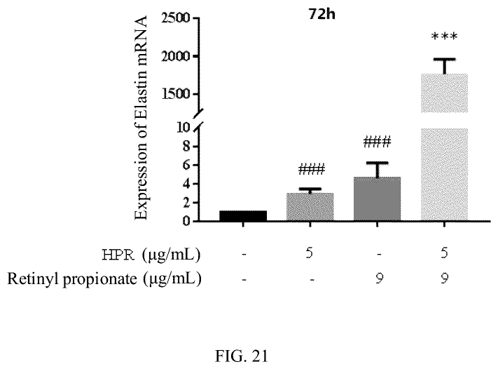

FIGS. 18-21 successively showed results of the effect on the expressions of the Col-IV mRNA, the CRBPI mRNA, the RARB mRNA, and Elastin mRNA after the HFF-1 cells were incubated with 5 μg/mL of the HPR and 9 μg/mL of the retinyl propionate alone or in combination for 72 h.

(In FIGS. 10-21: n=3, Mean±SEM. “*” indicated p<0.05 compared to the control group, “**” indicated p<0.01 compared to the control group, and “***” indicated p<0.001 compared to the control group; and “#” indicated p<0.05 compared to the HPR+retinyl propionate group, “##” indicated p<0.01 compared to the HPR+retinyl propionate group, and “###” indicated p<0.001 compared to the HPR+retinyl propionate group.)

The results of FIGS. 10-21 showed that the cells were administrated with 5 μg/mL of the HPR and 9 μg/mL of the retinyl propionate alone or in combination, total RNA was extracted at 24 h, 48 h, and 72 h, the expressions of the collagen-related mRNAs were detected by qPCR, and compared with the single administration of 5 μg/mL of the HPR and 9 μg/mL of the retinyl propionate, the combined administration of 5 μg/mL of the HPR and 9 μg/mL of the retinyl propionate can not only significantly promote the expression of the Elastin mRNA (the result of the Elastin expression comparison experiment was verified again), but also significantly promote the expressions of the Col-IV mRNA, the CRBPI mRNA, and the RARB mRNA.

In conclusion, it can be known from the experimental results that in the technical solution provided by the present disclosure, the HPR and the retinyl propionate were combined at a ratio of 5:9 to 20:9 in parts by mass to exert a synergistic effect, thereby obtaining an unexpected anti-aging effect. Particularly, when the HPR and the retinyl propionate were combined at a ratio of 5:9 in parts by mass, the anti-aging synergistic effect was particularly significant.

According to the technical solution provided by the present disclosure, the cooperation of the HPR and the retinyl propionate can extremely significantly increase the expression of the type IV collagen to 4 times or more. The type IV collagen is an important protein for combining an epidermal layer and a dermal layer, and links the two skin layers like a bridge. If the bridge structure is damaged, the epidermal layer and the dermal layer are not tightly linked, such that sagging is caused. The significant increase in the expression of the type IV collagen can effectively relieve the skin sagging problem.

According to the technical solution provided by the present disclosure, the cooperation of the HPR and the retinyl propionate can extremely significantly increase the expression of the CRBP-1 to 30 times or more. The CRBP-1 is a receptor after skin cells combine retinol and a derivative thereof. The increase of the expression amount represents that more effective retinol functions.

According to the technical solution provided by the present disclosure, the cooperation of the HPR and the retinyl propionate can extremely significantly increase the expression of the RARB to 5 times or more. The RARB is a retinoic acid receptor. After retinoic acid is bound to the receptor, the downstream effect of promoting collagen and elastin can be started. The increase of the expression of the receptor can significantly enhance the effects of the retinol-related substances.

According to the technical solution provided by the present disclosure, the cooperation of the HPR and the retinyl propionate can extremely significantly increase the expression of the Elastin to 300 times or more. The Elastin is a most important structural protein in the skin like collagen. The increase of the expression amount can significantly prevent aging.

Those of ordinary skill in the art should understand that the above embodiments are specific examples for implementing the present disclosure. In practical applications, various changes may be made to the above embodiments in terms of form and details without departing from the spirit and scope of the present disclosure.

What is claimed is:

1. A retinol derivative composition, comprising: hydroxypinacolone retinoate and retinyl propionate, wherein the ratio of the hydroxypinacolone retinoate to the retinyl propionate in parts by mass is (5-40):9.

2. The retinol derivative composition according to claim 1, wherein the ratio of the hydroxypinacolone retinoate to the retinyl propionate in parts by mass is (5-20):9.

3. The retinol derivative composition according to claim 1, wherein the ratio of the hydroxypinacolone retinoate to the retinyl propionate in parts by mass is (5-10):9.

4. The retinol derivative composition according to claim 1, wherein the ratio of the hydroxypinacolone retinoate to the retinyl propionate in parts by mass is 5:9.

5. A cosmetics and/or skin care product comprising the retinol derivative composition according to claim 1.

6. The cosmetics and/or skin care product according to claim 5, wherein the cosmetics and/or the skin care product is adapted to be applied to external surface of human skin.

7. The cosmetics and/or skin care product according to claim 5, wherein the cosmetics and/or the skin care product is one of an eye cream, a face cream, an essence, a lotion, and a facial mask essence.

8. The cosmetics and/or skin care product according to claim 5, wherein the retinol derivative composition is an additive dispersed in a matrix raw material, and the matrix raw material is selected from one or more of an oil raw material, a powder raw material, and a colloid raw material.

9. The cosmetics and/or skin care product according to claim 5, further comprising one or more of a surfactant, an essence, a spice, a pigment, a humectant, a preservative, an antioxidant, an ultraviolet absorber, a chelating agent, an astringent, a penetration enhancer, and a nutritional additive.

10. The cosmetics and/or skin care product according to claim 5, wherein the ratio of the hydroxypinacolone retinoate to the retinyl propionate in parts by mass is (5-20):9.

11. The cosmetics and/or skin care product according to claim 5, wherein the ratio of the hydroxypinacolone retinoate to the retinyl propionate in parts by mass is (5-10):9.

12. The cosmetics and/or skin care product according to claim 5, wherein the ratio of the hydroxypinacolone retinoate to the retinyl propionate in parts by mass is 5:9.
